(19) United States
(12) Patent Application Publication
(10) Pub. No.: US 2022/0204547 A1
(43) Pub. Date: Jun. 30, 2022

(54) NOVEL METHOD FOR SYNTHESIZING 25-OH CHOLESTEROL/CALCIFEDIOL FROM PHYTOSTEROL

(71) Applicant: FERMENTA BIOTECH LIMITED, Thane (West) (IN)

(72) Inventors: Anupama DATLA, Thane (West) (IN); Prashant Nagre, Thane (West) (IN); Jagdish Tamore, Thane (IN); Manojkumar Sadanand Prabhu, Thane (IN); Sochin Vasant Kadam, Thane (IN); Amoi Shirsath, Abernanth (IN)

(21) Appl. No.: 17/609,935

(22) PCT Filed: May 8, 2020
(86) PCT No.: PCT/IN2020/050416

§ 371 Date: Nov. 9, 2021

(30) Foreign Application Priority Data
May 9, 2019 (IN) 201921018561

(51) Int. Cl. C07J 9/00; C07C 29/62; C07C 29/09

(52) U.S. Cl. CPC C07J 9/00; C07C 29/095; C07C 29/62

(57) ABSTRACT

The present invention discloses novel method for synthesizing vegan 25-OH cholesterol/Calcifediol from inexpensive crude phytosterol. According to the method, Phytosterols are reacted to form corresponding i-steroid through tosylation and methanolysis. i-steroid on reductive ozonolysis to C-22 alcohol and conversion via C-22 tosylate to C-22 iodide in good yield. Coupling of C-22 tosylate with Grignard reagent of 4-bromo-2-methyl-2-[(trimethylsilyl)oxy]butane followed by deprotection yielded 25-OH cholesterol. In a process variant, nickel mediated conjugate addition of C-22 iodide to an electron deficient alkene ethyl acrylate and treating corresponding ester with methyl magnesium bromide as means of installing the side chain of 25-OH cholesterol in high yield. Further bromination reaction of 25-OH cholesterol diacetate followed by dehydrobromination using TBAF yielded 25-OH 7-dehydrocholesterol. Further photo reaction of 25-OH 7-dehydrocholesterol in to previtamin D3 using high or medium pressure mercury lamp and subsequent thermal reaction of previtamin D3 to 25-OH vitamin D3 (Calcifediol) in good yield.

### TECHNICAL FIELD

The present invention relates to novel method for synthesizing vegan 25-OH cholesterol/Calcifediol from inexpensive crude phytosterol.

### BACKGROUND AND PRIOR ART

The metabolite of vitamin D3, 25-hydroxycholecalciferol (Calcifediol) is more potent anti-rachitic agent than vitamin D itself therefore development of a facile method for the synthesis of vitamin D3 and its analogue Calcifediol is highly important. Further, 25-hydroxy cholesterol is an important raw material for the synthesis of 25-hydroxy vitamin D3, also known as calcifediol, is the active metabolite of vitamin D3, has a stronger physiological activity, and does not need to go through the liver metabolism.

The 25-hydroxy vitamin D3 has the following unique functions. Vitamin D3 in some human or animals cannot be directly converted into 25-hydroxy vitamin D3 due to liver function disorder. Since the 25-hydroxy vitamin D3, as an active substance, bypasses the liver transformation and hence can be directly supplied for human or animals for absorption. The 25-hydroxy vitamin D3 in animals can promote the bone development of poultry, maximize the bone mineral density, reduce chick mortality, reduce osteoporosis and cage layer fatigue, improve quality of egg shells, reduce the breakage rate of egg shells, increase the hatching rate and prolong the egg production cycle. When compared with vitamin D3, absorption of the 25-hydroxy vitamin D3 is less affected by intestinal damage; the content of the 25-hydroxy vitamin D3 in plasma is a direct indicator for the nutritional status of vitamin D3.

Although, the market requirement of 25-hydroxy cholesterol is more, however, the availability of the same is less due to the difficulty in the existing production processes.

The preparation of 25-hydroxycholecalciferol had been reported from 25-hydroxy cholesterol which in turn is prepared from pure stigmasterol which is again isolated from crude phytosterol. Phytosterol is a mixture of sterols i.e. a mixture of stigmasterol, beta-sitosterol, campesterol and stigmastenol.

Stigmasterol is an unsaturated sterol, having one double bond in the sterol ring structure and one double bond in the side chain. Beta sitosterol is an unsaturated sterol with one double bond in sterol ring structure. Campesterol is structurally similar to beta sitosterol but has methyl group substituent at C24 position instead of an ethyl group. Stigmastenol is a saturated sterol both in ring structure and in the side chain.

There is very limited literature available on methods of preparation of 25-hydroxycholesterol and its esters starting from the naturally occurring phytosterol. U.S. Pat. No. 3,822,254 discloses a process for the preparation of 25-hydroxycholesterol and its esters starting from the naturally occurring (readily available and inexpensive) starting material, stigmasterol which is isolated commercially from Soybeans. The synthesis encompasses, as key steps, the protection of the 3-hydroxy-delta 5-function by formation of an i-steroid, cleavage of the 22, 23-double bond, and introduction of the properly substituted 5-carbon fragment to afford the 25-hydroxycholesterol side chain. The synthesis of 25-hydroxycholesterol from pure stigmasterol as reported in U.S. Pat. No. 3,822,254 is shown in scheme 1.

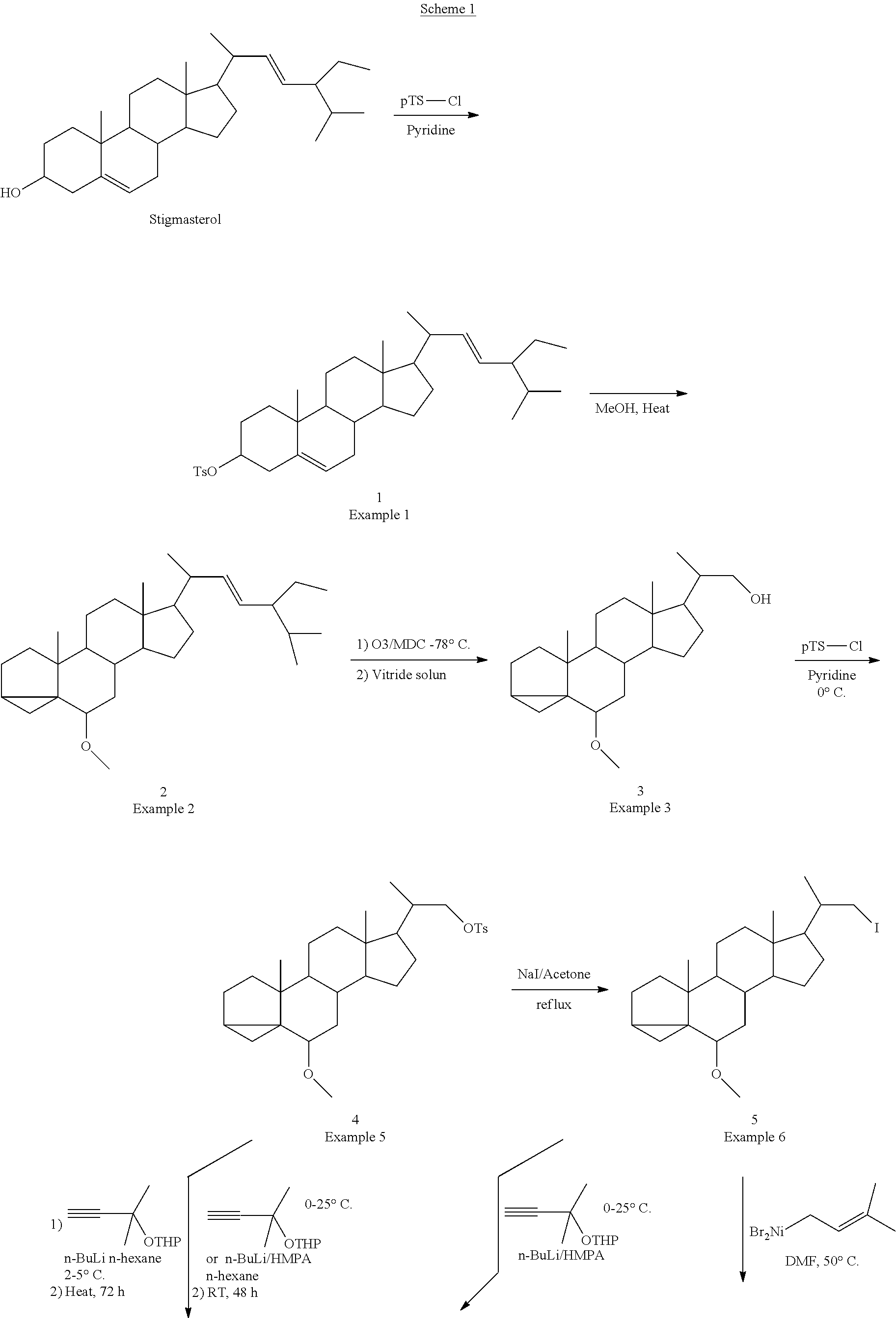

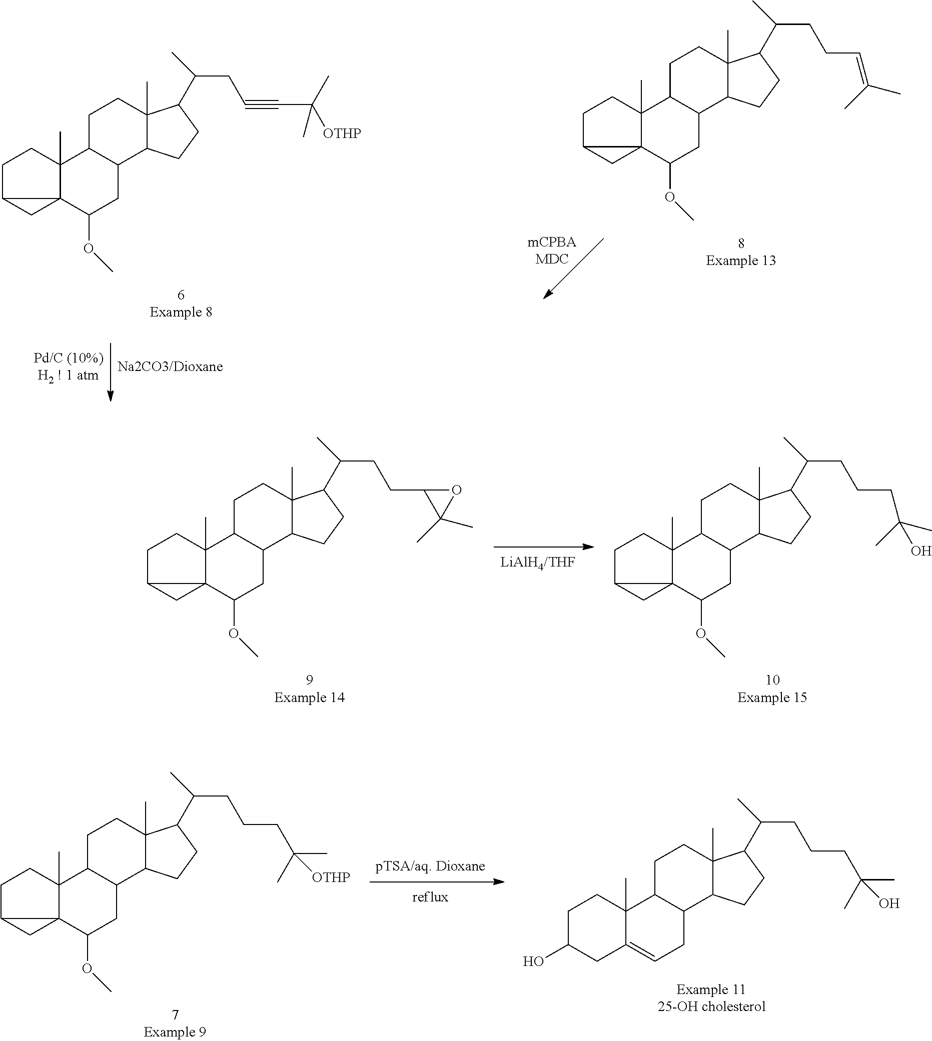

The reported separations of pure stigmasterol from phytosterol are relatively laborious and cumbersome in view of close structural relationship of stigmasterol with other sterols in the mixture viz., beta-sitosterol, campesterol and stigmastenol.

There is literature available for the preparation of 25hydroxycholesterol and esters thereof other than phytosterol.

AU1034095 discloses a process for producing a 25-hydroxycholesterol by hydroxylating cholesterol at the 25-position using ruthenium compound as a catalyst, however, the source of cholesterol is not disclosed.

U.S. Pat. No. 3,846,455A discloses a process for the preparation of 25hydroxycholesterol and esters thereof, which comprises, a) treating fucosterol-24(28)-epoxide or esters thereof with stannic chloride; b) the formation of desmosterol-24(25)-epoxide or 3-esters thereof by treating the desmosterol or esters thereof obtained by the preceding step, with a per organic acid; and, c) the formation of 25-hydroxycholesterol or its 3-ester as the final product by treating the desmosterol-24(25)-epoxide or 3-ester thereof obtained by the above step (b), with a complex compound of an alkali metal hydride and, if desired, further reacting the resulting 25-hydroxycholesterol with an esterifying agent.

U.S. Pat. No. 4,183,852A discloses synthesis of 25-hydroxycholesterol and 25-hydroxycholecalciferol from animal bile starting materials in which hyodeoxycholic acid or an ester thereof is converted to the 30-hydroxy-5-cholenic acid alkyl ester, and this is converted to 30-hydroxy-25-cyano-5-cholene by a series of steps by which the sterol nucleus is stabilized by placing a protecting group at the 3 position and then extending the chain from the carbon at the 24 position to a cyanide group at the 25 position. The compound so formed is subjected to a series of reactions by which it is transformed into 25-hydroxy-7-dehydrocholesterol which may then be irradiated with ultraviolet light to 25-hydroxycholecalciferol.

QianZhao et al report synthesis of 25-hydroxycholesterol from desmosterol (Steroids, Volume 85, July 2014, Pages 1-5). The desmosterol was brominated in THF-water (4:1, v/v) using NBS as the brominating agent, followed by the reduction of C—Br with lithium aluminum hydride in THF, to obtain product, 25-hydroxycholesterol with yields and regioselectivity. The reaction is shown in scheme 2 below.

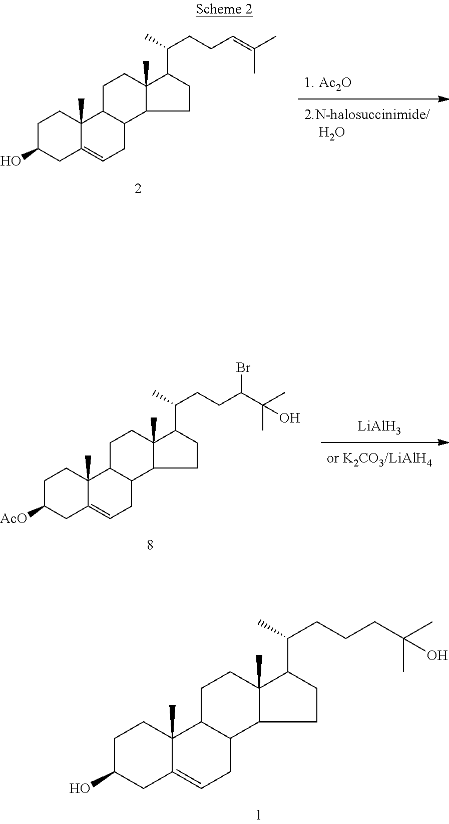 [SMILES: CC(C)=CCC[C@@H](C)C1CCC2C3CC=C4C[C@@H](O)CC[C@]4(C)C3CC[C@@]21C ; CC(=O)O[C@H]1CC[C@@]2(C)C(=CCC3C4CCC([C@H](C)CCC(Br)C(C)(C)O)[C@@]4(C)CCC32)C1 ; "LiAlH3" ; C[C@H](CCCC(C)(C)O)C1CCC2C3CC=C4C[C@@H](O)CC[C@]4(C)C3CC[C@@]21C]

Another method is reported in IN 201717006223, for synthesizing 25-hydroxy cholesterol, characterized in that: adding a 24-dehydrocholesterol derivative and a hydroxyl containing reagent to an organic solvent and react at −40° C.˜150° C. for 1˜40 hours in the presence of a catalyst; after completing the reaction, 5 hydrolyzing with alkali, and then separating the reaction solution to obtain 25-hydroxy cholesterol, wherein the structural formulas of the 25-hydroxy cholesterol, the 24-dehydrocholesterol derivative and the hydroxyl containing reagent are shown in formula (I), formula (II) and formula (III) respectively:

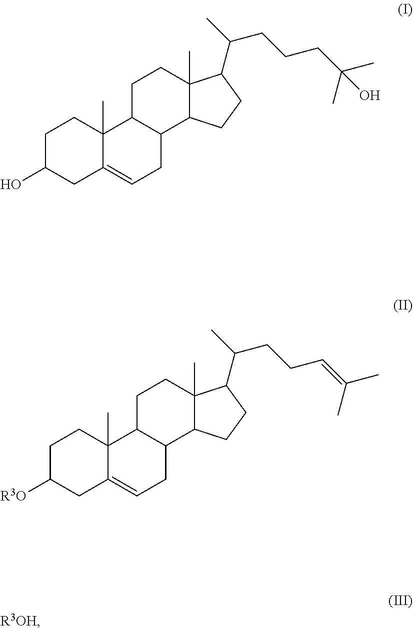

wherein, the R1 in formula (II) is the same as the R2 in formula (III), which is H or C1-C12 acyl group.

Plant sterols and plant stanols, known commonly as phytosterols, are plant-derived compounds that are structurally related to cholesterol and abundant in nature and hence the synthesis of 25-OH cholesterol/Calcifediol from phytosterol will be cost-effective as it is readily available and inexpensive.

Therefore, the objective of the present invention is to provide a cost-effective and robust process for the preparation of 25-OH cholesterol/Calcifediol from phytosterol.

### SUMMARY OF THE PRESENT INVENTION

In line with the above objective, the present invention provides a process for preparation of 25-OH Vit D3 (Calcifediol) which comprises;
a) Reacting (20S)-20-hydroxymethyl-6β-methoxy-3α,5-cyclo-5α-pregnane (3) with Iodine and imidazole in presence of triphenyl phosphine to obtain (20S)-20-iodo methyl-6β-methoxy-3α,5-cyclo-5a-pregnane (5);
b) Reacting the (20S)-20-iodo methyl-6β-methoxy-3α,5-cyclo-5α-pregnane (5) with ethyl acrylate in presence of a reducing agent and a base to obtain Ethyl 1(S)-6β-methoxy-3α,5-cyclo-5α-cholesta-25-oate (6);
c) Reacting the Ethyl 1(S)-6β-methoxy-3α,5-cyclo-5α-cholesta-25-oate (6) with methyl magnesium bromide in a solvent, at room temperature under N₂ followed by treating with a dehydrating agent in a solvent to obtain 25-OH cholesterol (7);
d) Reacting the 25-OH cholesterol (7) with acetic anhydride in presence of a base at 30-50° C. to obtain 25-OH cholesterol diacetate (8);
e) Brominating the 25-OH cholesterol diacetate with brominating agent in presence of an initiator at 50 to 80° C. to obtain 25-OH 7-bromo cholesteryl diacetate;
f) Treating the 25-OH 7-bromo cholesteryl diacetate with TBAF in a solvent at 10 to 30° C. to obtain 25-OH 7-dehydrocholesteryl diacetate followed by subjecting the 25-OH 7-dehydrocholesteryl diacetate to alkaline hydrolysis at about 3 to 60° C. to obtain 25-OH 7-dehydrocholesterol (9);
g) Irradiating the 25-Hydroxy-7-dehydrocholesterol (9) in a solvent under high pressure mercury lamp in presence of a sensitizer to obtain 25-hydroxy-pre vitamin D3; and
h) Heating the solution containing 25-hydroxy-pre D3 to convert 25-hydroxy-pre D3 to 25-OH vitamin D3 (Calcifediol).

In another aspect, the invention provides a process for preparation of 25-OH cholesterol (7) comprising the steps of:
a) Reacting (20S)-20-hydroxymethyl-60-methoxy-3α,5-cyclo-5α-pregnane (3) with Iodine and imidazole in presence of triphenyl phosphine to obtain (20S)-20-iodo methyl-6β-methoxy-3α,5-cyclo-5α-pregnane (5);
b) Reacting the (20S)-20-iodo methyl-6β-methoxy-3α,5-cyclo-5α-pregnane (5) with ethyl acrylate in presence of a reducing agent zinc dust, nickel chloride and a base pyridine to obtain Ethyl 1(S)-60-methoxy-3α,5-cyclo-5α-cholesta-25-oate (6); and
c) Reacting the Ethyl 1(S)-60-methoxy-3α,5-cyclo-5α-cholesta-25-oate (6) with methyl magnesium bromide in a solvent at room temperature under N2 followed by treating with a dehydrating agent in a solvent to obtain 25-OH cholesterol (7).

In a process variant, the invention provides process for preparation of 25-OH cholesterol which process comprises;
a) Preparing a solution of magnesium turnings in presence of 1,2-dibromoethane and 4-bromo-2-methyl-2-[(trimethylsilyl)oxy]butane in a solvent at 40 to 60° C. under N2;
b) Reacting the contents of step a) with (20S)-60-methoxy-20-(p-toluene sulfonoxymethyl)-3α, 5-cyclo-5α-pregnane (4) and CuBr.Me2S solution in a solvent to obtain 25-OH cholesterol (7).

In yet another aspect, the invention provides a process for preparation of 25-OH Vitamin D3 (Calcifediol) from 25-OHcholesterol, which process comprises;
a) Reacting 25-OH cholesterol (7) with acetic anhydride in presence of a base at 30-50° C. to obtain 25-OH cholesterol diacetate (8);
b) Brominating the 25-OH cholesterol diacetate with DDH (1,3-dibromo-5,5-dimethyl hydantoin) in presence of an initiator at 50 to 80° C. to obtain 25-OH 7-bromo cholesteryl diacetate;
c) Treating the 25-OH 7-bromo cholesteryl diacetate with TBAF in a solvent at 10 to 30° C. to obtain 25-OH 7-dehydrocholesteryl diacetate followed by subjecting the 25-OH 7-dehydrocholesteryl diacetate to alkaline hydrolysis at about 30 to 60° C. to obtain 25-OH 7-dehydrocholesterol (9);
d) Irradiating the 25-Hydroxy-7-dehydrocholesterol (9) in THF under high pressure mercury lamp in presence of a sensitizer to obtain 25-hydroxy-pre D3; and
e) Heating the solution containing 25-hydroxy-pre D3 to convert 25-hydroxy-pre D3 to 25-OH vitamin D3 (Calcifediol).

In a further aspect, the invention provides a process for preparation of 25-OH-7-dehydrocholesterol (9) which process comprises;
a) Reacting 25-OH cholesterol (7) with acetic anhydride in presence of base at 30-50° C. to obtain 25-OH cholesterol diacetate (8);
b) Brominating the 25-OH cholesterol diacetate with DDH(1,3-dibromo-5,5-dimethyl hydantoin) in presence of an initiator at 50 to 80° C. to obtain 25-OH 7-bromo cholesteryl diacetate; and
c) Treating the 25-OH 7-bromo cholesteryl diacetate with TBAF in a solvent at 10 to 30° C. to obtain 25-OH 7-dehydrocholesteryl diacetate followed by subjecting the 25-OH 7-dehydrocholesteryl diacetate to alkaline hydrolysis at about 30 to 60° C. to obtain 25-OH 7-dehydrocholesterol (9).

### DETAILED DESCRIPTION

The invention will now be described in detail in connection with certain preferred and optional embodiments, so that various aspects thereof may be more fully understood and appreciated.

Accordingly the present invention provides process for preparation of 25-OH cholesterol, 25-OH 7-dehydrocholesterol and finally Calcifediol directly from readily available inexpensive crude phytosterol, as shown in schemes 3 and 4.

Preparation of Phytosteryl tosylate (1); Phytosteryl-1-methyl ether (2); (20S)-20-hydroxymethyl-6β-methoxy-3α,5-cyclo-5α-pregnane (3); (20S)-6β-methoxy-20-(p-toluene sulfonoxy methyl)-3α,5-cyclo-5α-pregnane (4), starting from crude phytosterol as demonstrated in examples 1 to 4, are prepared as per teachings of U.S. Pat. No. 3,822,254.

In an embodiment, the invention provides a process for preparation of 25-OH Vit D3 (Calcifediol), which comprises;
a) Reacting (20S)-20-hydroxymethyl-6β-methoxy-3α,5-cyclo-5α-pregnane (3) with Iodine and imidazole in presence of triphenyl phosphine to obtain (20S)-20-iodo methyl-6β-methoxy-3α,5-cyclo-5a-pregnane (5);
b) Reacting the (20S)-20-iodo methyl-6β-methoxy-3α,5-cyclo-5a-pregnane (5) with ethyl acrylate in presence of a reducing agent and a base to obtain Ethyl 1(S)-6β-methoxy-3α,5-cyclo-5α-cholesta-25-oate (6);
c) Reacting the Ethyl 1(S)-60-methoxy-3α,5-cyclo-5α-cholesta-25-oate (6) with methyl magnesium bromide in a solvent, at room temperature under N₂ followed by treating with a dehydrating agent in a solvent to obtain 25-OH cholesterol (7);
d) Reacting the 25-OH cholesterol (7) with acetic anhydride in presence of a base at 30-50° C. to obtain 25-OH cholesterol diacetate (8);
e) Brominating the 25-OH cholesterol diacetate with brominating agent in presence of an initiator at 50 to 80° C. to obtain 25-OH 7-bromo cholesteryl diacetate;
f) Treating the 25-OH 7-bromo cholesteryl diacetate with TBAF in a solvent at 10 to 30° C. to obtain 25-OH 7-dehydrocholesteryl diacetate followed by subjecting the 25-OH 7-dehydrocholesteryl diacetate to alkaline hydrolysis at about 30 to 60° C. to obtain 25-OH 7-dehydrocholesterol (9);
g) Irradiating the 25-Hydroxy-7-dehydrocholesterol (9) in a solvent under high pressure mercury lamp in presence of a sensitizer to obtain 25-hydroxy-pre vitamin D3; and
h) Heating the solution containing 25-hydroxy-pre D3 to convert 25-hydroxy-pre D3 to 25-OH vitamin D3 (Calcifediol).

The reaction in step b) is carried out at a temperature range of 40 to 70° C.

In a preferred embodiment, the reducing agent in step b) is combination of zinc dust and nickel chloride. However, any other suitable reducing agents such as sodium borohydride, sodium hydride, metal catalysts such as palladium or platinum catalysts etc. can be employed to achieve the desired result.

The heating in step c) is carried out at a temperature range of 50 to 90° C.

In a preferred embodiment, the dehydrating agent in step c) is para toluene sulphonic acid. However, any other suitable dehydrating agents such as sulphuric acid, phosphorous pentoxide, calcium oxide, orthoformic acid, etc. can be employed to achieve the desired result.

The base may be selected from organic or inorganic base. The base is preferably an organic base selected from pyridine, DMAP or combinations thereof. However, any other suitable organic or inorganic bases can be employed to achieve the desired result.

The solvent may be selected from THF, diethylether, dioxane or any other suitable solvents such as acetone, ethylacetate, methylene chloride, ethylene dichloride, acetonitrile, toluene, xylene etc.

In a preferred embodiment, the initiator in step e) is Bis(tert-butylcyclohexyl) peroxydicarbonate and the brominating agent is DDH(1,3-dibromo-5,5-dimethyl hydantoin). However, any other suitable initiators such as AIBN and brominating agents such as NBS can be employed to achieve the desired result.

In a preferred embodiment, the irradiation sensitizer used in step g) is 5-(3-pyridyl)-2,2′-bithiophene. However, any other suitable irradiation sensitizer such as anthracene, 9-acetyl anthracene can be employed to achieve the desired result. In a preferred embodiment, the solvent is selected from ethers such as THF, diethylether, dioxane. However, any other suitable solvents can be employed to achieve the desired result.

The process for preparation of 25-OH vitamin D3(CalcWfediol) is shown below in scheme 3.

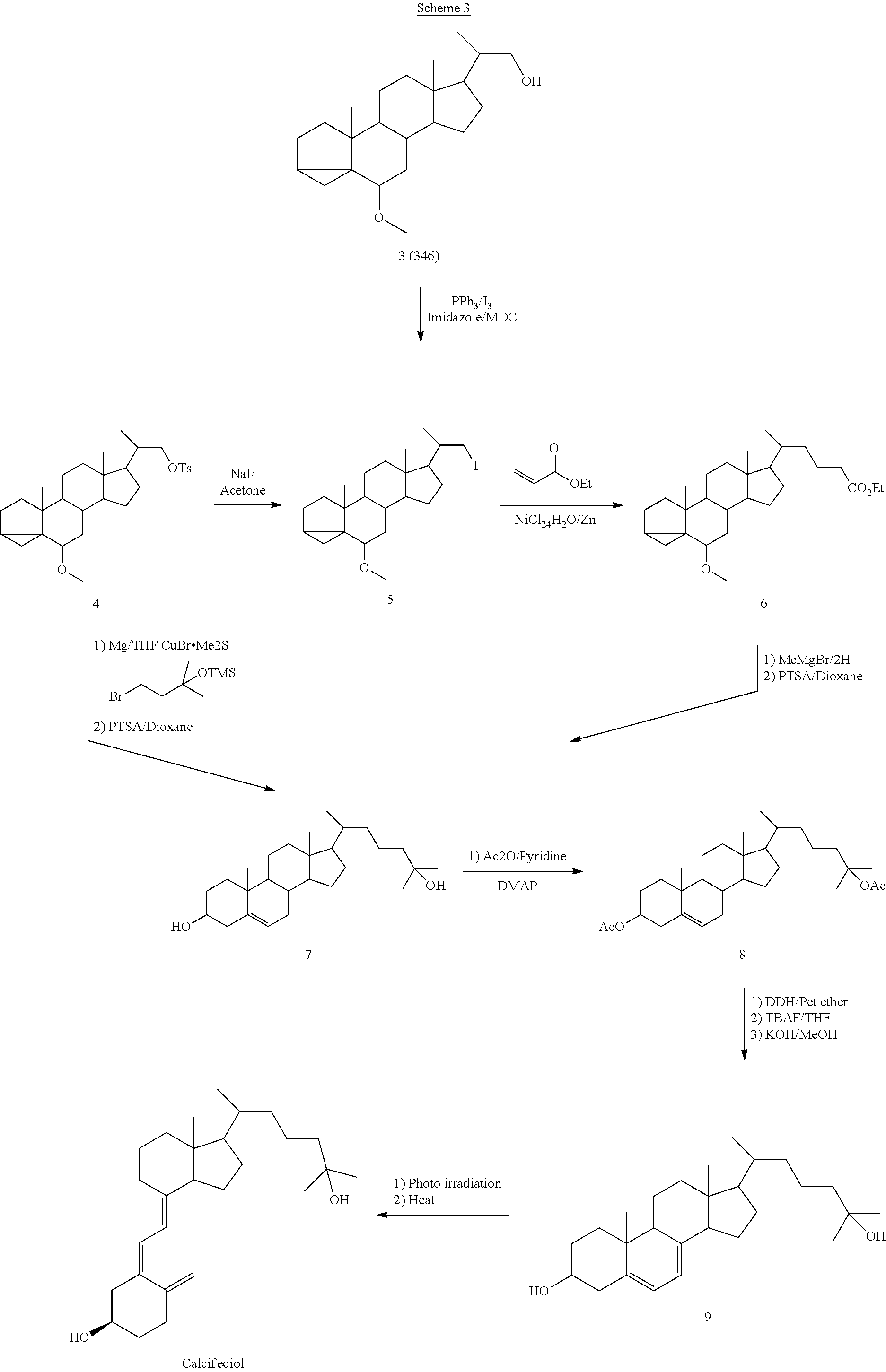

In another embodiment, the invention provides a process for preparation of 25-OH cholesterol (7) comprising the steps of:
a) Reacting (20S)-20-hydroxymethyl-60-methoxy-3α,5-cyclo-5α-pregnane (3) with Iodine and imidazole in presence of triphenyl phosphine to obtain (20S)-20-iodo methyl-6β-methoxy-3α,5-cyclo-5a-pregnane (5);
b) Reacting the (20S)-20-iodo methyl-6β-methoxy-3α,5-cyclo-5α-pregnane (5) with ethyl acrylate in presence of a reducing agent zinc dust, nickel chloride and a base pyridine to obtain Ethyl 1(S)-60-methoxy-3α,5-cyclo-5α-cholesta-25-oate (6); and
c) Reacting the Ethyl 1(S)-60-methoxy-3α,5-cyclo-5α-cholesta-25-oate (6) with methyl magnesium bromide in a solvent at room temperature under N2 followed by treating with a dehydrating agent in a solvent to obtain 25-OH cholesterol (7).

The reaction in step b) is carried out at a temperature range of 40 to 70° C.

The reducing agent in step b) is combination of zinc dust and nickel chloride.

The heating in step c) is carried out at a temperature range of 50 to 90° C.

The base in steps b) and step d) is selected from pyridine, DMAP or combinations thereof.

The process the dehydrating agent in step c) is para toluene sulphonic acid.

In a process variant, the invention discloses a process for preparation of 25-OH cholesterol (7) which comprises:
a) Preparing a solution of magnesium turnings in presence of 1,2-dibromoethane and 4-bromo-2-methyl-2-[(trimethylsilyl)oxy]butane in a solvent at 40 to 60° C. under N2;
b) Reacting the contents of step a) with (20S)-60-methoxy-20-(p-toluene sulfonoxymethyl)-3α, 5-cyclo-5α-pregnane (4) and CuBr.Me2S solution in a solvent to obtain 25-OH cholesterol (7).

In an embodiment, the solvent is selected from THF, diethylether, dioxane. However, any other suitable solvents can be employed to achieve the desired result.

The reaction scheme for the synthesis of 25-OH-cholesterol is shown in scheme 4.

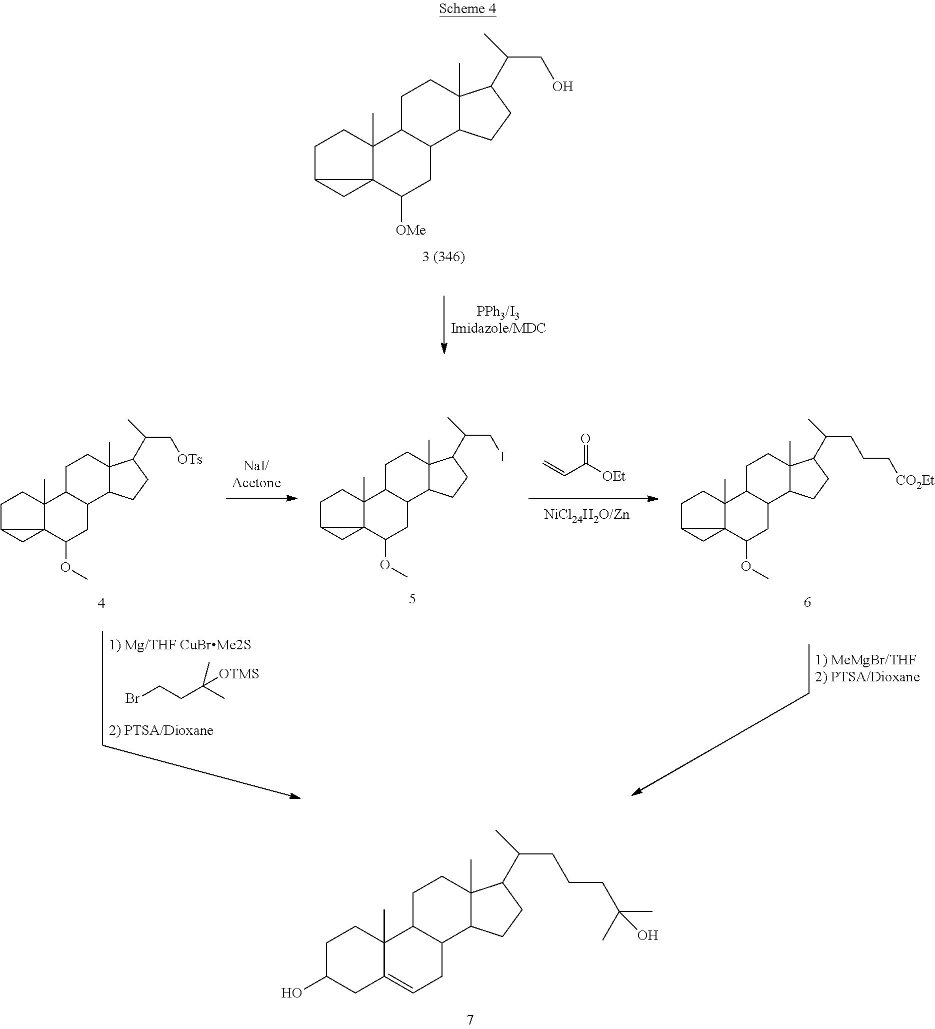

In yet another embodiment, the invention provides a process for preparation of 25-OH Vitamin D3 (Calcifediol) from 25-OHcholesterol (scheme 3), which process comprises;
a) Reacting 25-OH cholesterol (7) with acetic anhydride in presence of a base at 30-50° C. to obtain 25-OH cholesterol diacetate (8);
b) Brominating the 25-OH cholesterol diacetate with DDH(1,3-dibromo-5,5-dimethyl hydantoin) in presence of an initiator at 50 to 80° C. to obtain 25-OH 7-bromo cholesteryl diacetate;
c) Treating the 25-OH 7-bromo cholesteryl diacetate with TBAF in a solvent at 10 to 30° C. to obtain 25-OH 7-dehydrocholesteryl diacetate followed by subjecting the 25-OH 7-dehydrocholesteryl diacetate to alkaline hydrolysis at about 30 to 60° C. to obtain 25-OH 7-dehydrocholesterol (9);
d) Irradiating the 25-Hydroxy-7-dehydrocholesterol (9) in THF under high pressure mercury lamp in presence of a sensitizer to obtain 25-hydroxy-pre D3; and
e) Heating the solution containing 25-hydroxy-pre D3 to convert 25-hydroxy-pre D3 to 25-OH vitamin D3 (Calcifediol).

The base is selected from pyridine, DMAP or combinations thereof and the solvent is selected from THF, diethylether, dioxane.

The irradiation sensitizer used in step d) is selected from 5-(3-pyridyl)-2,2′-bithiophene, anthracene and 9-acetyl anthracene.

The initiator used in the above process is selected from Bis(tert-butylcyclohexyl) peroxydicarbonate or AIBN and the brominating agent is selected from DDH(1,3-dibromo-5,5-dimethyl hydantoin) and NBS.

In a further embodiment, the invention provides a process for preparation of 25-OH-7-dehydrocholesterol (9) which process comprises;
a) Reacting 25-OH cholesterol (7) with acetic anhydride in presence of base at 30-50° C. to obtain 25-OH cholesterol diacetate (8);
b) Brominating 25-OH cholesterol diacetate with DDH(1,3-dibromo-5,5-dimethyl hydantoin) in presence of an initiator at 50 to 80° C. to obtain 25-OH 7-bromo cholesteryl diacetate; and
c) Treating the 25-OH 7-bromo cholesteryl diacetate with TBAF in a solvent at 10 to 30° C. to obtain 25-OH 7-dehydrocholesteryl diacetate followed by subjecting the 25-OH 7-dehydrocholesteryl diacetate to alkaline hydrolysis at about 30 to 60° C. to obtain 25-OH 7-dehydrocholesterol (9).

The base may be selected from organic or inorganic base. The base is preferably an organic base selected from pyridine, DMAP or combinations thereof and the solvent is selected from THF, diethylether, dioxane or any other suitable solvents such as acetone, ethylacetate, methylene chloride, ethylene dichloride, acetonitrile, toluene, xylene etc.

The initiator is selected from Bis(tert-butylcyclohexyl) peroxydicarbonate or AIBN and the brominating agent is selected from DDH (1,3-dibromo-5,5-dimethyl hydantoin) or NBS.

In yet another embodiment, the invention encompasses a novel intermediate compound of formula 6, i.e., Ethyl 1(S)-60-methoxy-3α,5-cyclo-5α-cholesta-25-oate, structurally as shown below:

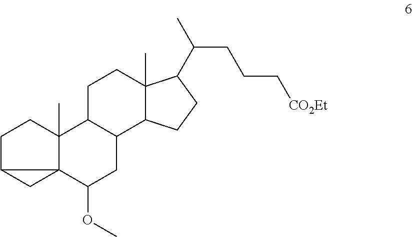 [SMILES: CCOC(=O)CCCC(C)C1CCC2C3CC(OC)C45CC4CCC5(C)C3CCC12C]

In yet another embodiment, the invention encompasses a novel intermediate compound of formula (8), i.e, 25-OH cholesteryl diacetate, structurally as shown below:

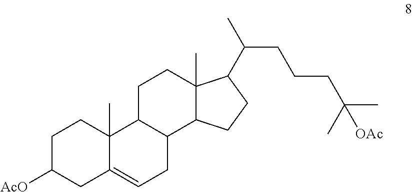 [SMILES: CC(=O)OC1CCC2(C)C(=CCC3C2CCC2(C)C(C(C)CCCC(C)(C)OC(C)=O)CCC32)C1]

In yet another embodiment, the invention encompasses a novel intermediate compound of formula (9), i.e, 25-OH 7-dehydrocholesterol, structurally as shown below:

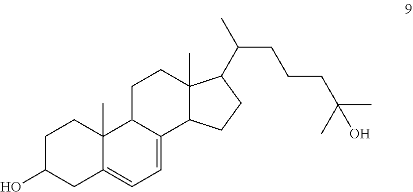 [SMILES: CC(CCCC(C)(C)O)C1CCC2C3=CC=C4CC(O)CCC4(C)C3CCC21C]

The compound 6, 8 and 9 are further characterised by NMR, IR, Mass spectroscopy.

The invention further encompasses novel intermediate, 25-OH 7-bromo cholesteryl diacetate, however is not isolated due to instability of this compound.

The present invention is exemplified by the following examples which are provided for illustration only and, should not be construed to limit the scope of the invention.

### EXAMPLES

### Example 1

Preparation of Phytosterol Tosylate (1)

To a solution of 500.0 g (1.20 mole) of Phytosterol in 5000 ml of dry pyridine was added 500.0 g (2.62 mole) of p-toluene sulfonyl chloride and the mixture was stirred at 25° C. for 16 hrs. Pyridine was removed by vacuum distillation and the residue was slowly poured into 10% sodium carbonate solution. The precipitated product was collected by filtration, washed with water followed by methanol and dried in vacuum overnight to yield 600.0 g. of phytosteryl tosylate used for next step without further purification.

Yield: 600 g (88%)

Appearance: White solid

GC analysis: Stigmasteryl tosylate: 20.37% (RT: 6.10)
Sitosteryl tosylate: 42.29% (RT: 6.80)
Campesteryl tosylate: 15.60% (RT: 5.76)

### Example 2

Preparation of Phytosterol-1-Methyl Ether (2)

A mixture of 600.0 g (1.06 mole) of phytostery tosylate in 5500 ml of methanol and 300 g (3.79 mole) of pyridine was stirred at 55° C. for 5 hrs. The cooled solution was concentrated under reduced pressure. The residue was poured into water and extracted with dichloromethane. The dichloromethane solution was dried over anhydrous sodium sulfate and evaporated to dryness to yield 420.0 g. of colorless thick oil used for next step without further purification.

Yield: 420 g (93%)

Appearance: colorless thick oil

GC analysis: Stigmasteryl-1-methyl ether: 19.49% (RT: 5.47)
Sitosteryl-1-methyl ether: 48% (RT 6.05)
Campesteryl-1-methylether: 15.08% (RT: 6.34)

### Example 3

Preparation of (20S)-20-hydroxymethyl-6β-methoxy-3α,5-cyclo-5α-Pregnane (3)

A solution of 420.0 g (0.98 mole) of phytosteryl-1-methyl ether in 4000 ml of methylene chloride and 1300 ml of methanol was cooled to −78° C. and treated with ozonized oxygen for 3-4 h. The reaction vessel was flushed with nitrogen and 42 g (1.11 mole) of sodium borohydride was added. The mixture was stirred at −50° C. for 1 h and then allowed to warm to 0° C. over a 1 h period. Water was added slowly to decompose the excess hydride and the product was extracted with methylene chloride. The methylene chloride solution was washed with brine solution. The methylene chloride solution was then dried over anhydrous sodium sulfate and evaporated to dryness. The 400 g of crude reaction mass was purified by column chromatography using silica gel to get 60.0 g. of (20S)-20-hydroxy methyl-60-methoxy-3α,5-cyclo-5α-pregnane.

Yield: 60 g (90%)

Appearance: colorless solid

GC analysis: 93.6% purity (RT 3.35)

Fractions containing sitosterol-1-methyl ether collected separately and concentrated to get thick oil which upon heating in aqueous dioxane in presence of catalytic PTSA at 100° C. for 2h, followed by removal of solvent and crystallization in methanol gave 230 g of colourless solid as stigmasterol free form phytosterol.

Yield: 230 g (90%)

Appearance: colorless solid

GC analysis: >95% purity

### Example 4

### Preparation of (20S)-6β-methoxy-20-(p-toluene sulfooxy methyl)-3α,5-cyclo-5α-pregnane (4)

To a solution of 60 g (0.172 mole) of (20S)-20 hydroxymethyl-60-methoxy-3α,5-cyclo-5α-pregnane in 600 ml. of pyridine was added slowly 60 g. (0.314 mole) of p-toluene sulfonyl chloride at 0° C. The mixture was stirred at 0° C. for 5 h. Several chips of ice were added and the mixture was stirred for 5 minutes to decompose the excess p-toluene sulfonyl chloride. The mixture was poured into water and the product was extracted with methylene chloride. The methylene chloride solution was washed with water and brine solution. The solution was dried over anhydrous sodium sulfate and evaporated to dryness to yield 78.0 g white solid of (20S)-60-methoxy-20-(p-toluene sulfonoxy methyl)-3α, 5-cyclo-5α-pregnane used for next step without further purification,

Yield: 78 g (90%)

Appearance: colorless solid

### Example 5

### Preparation of (20S)-20-iodomethyl-6β-methoxy-3α,5-cyclo-5α-pregnane(5)

A mixture of 78.0 g. (0.156 mole) of (20S)-60-methoxy-20-(p-toluene sulfonoxy methyl)-3α, 5-cyclo-5α-pregnane, 44.9 g. (0.300 mole) of sodium iodide and 800 ml of dry acetone was heated at reflux for 3 hrs and cooled. The mixture was poured into water and extracted with ethyl acetate. The ethyl acetate extract was dried over anhydrous sodium sulfate to yield 71.0 g of pale yellow solid of (20S)-20-iodo methyl-6β-methoxy-3α,5-cyclo-5α-pregnane.

Yield: 71 g (98%)

Appearance: Pale yellow solid.

M.pt: 102-104° C. (rep: 103-104° C.)

GC analysis: 95% purity (RT: 6.03)

Alternatively (20S)-20-iodomethyl-60-methoxy-3α,5-cyclo-5α-pregnane (5) was synthesized directly from (20S)-20-hydroxy methyl-60-methoxy-3α,5-cyclo-5α-pregnane (3) as shown below in example 6.

### Example 6

### Preparation of (20S)-20-iodomethyl-6β-methoxy-3α,5-cyclo-5α-pregnane (5)

Iodine (28.7 g, 0.228 mol) was added to a stirred, cooled (0° C.) solution of 59 g (0.86 mol) of imidazole and 60 g (0.228 mol) of triphenylphosphine in 500 mL of CH2Cl2. The mixture was stirred for 15 min and treated with a solution of 50.0 g (0.144 mol) of (20S)-20-hydroxy methyl-6β-methoxy-3α,5-cyclo-5α-pregnane (3) in 250 mL of CH2Cl2 during 20 min, keeping the temperature below 10° C. Stirring was continued at 5° C. for 0.5 h and at room temperature for 2.0 h, and the mixture was filtered. The filter cake was washed with 100 mL of CH2Cl2, and the combined filtrate and washing were washed with 400 mL of 2% sodium thiosulfate, 300 mL of 0.1 N HCl, and 300 mL of brine, dried (over Na2SO4), and evaporated to give a pale yellow semisolid. This was stirred with 1.0 L of Et20 and filtered (to remove most triphenylphosphine oxide) and the filtrate was evaporated to get 62.5 g of off white solid.

Yield: 62.5 g (94%)

Appearance: Off white solid

M.Pt: 102-105° C. (rep: 103-104° C.)

GC analysis: 98.2% purity (RT: 6.03)

### Example 7

### Preparation of 25-OHcholesterol (7) from (20S)-6β-methoxy-20-(p-toluene sulfonoxymethyl)-3α,5-cyclo-5α-pregnane(4)

To stirred magnesium turnings (18.7 g, 0.78 mol) in THF (750 ml) few drops of 1,2-dibromoethane was added under Nitrogen atmosphere followed by few drops of 4-bromo-2-methyl-2-[(trimethylsilyl)oxy]butane and heated the mixture to 50° C. for few minutes to initiate reaction then a remaining solution of 4-bromo-2-methyl-2-[(trimethylsilyl)oxy]butane (312 g, 1.30 mol) in THF (250 mL) was added drop wise under N2. After being stirred at the same temperature 50° C. for 30 min, the reaction mixture was cooled at 0° C. and a suspension of CuBr.Me2S (4.0 g, 0.01 mol) was added and a solution of (20S)-60-methoxy-20-(p-toluene sulfonoxy methyl)-3α,5-cyclo-5α-pregnane (4) (78 g, 0.156 mol) in THF (250 mL) was added drop wise at 0° C. under N2. After being stirred at room temperature for 2-3 h, the reaction mixture was poured into saturated aqueous NH4Cl at 0° C. and the aqueous layer was extracted twice with EtOAc. The combined organic layer was washed with saturated aqueous NH4Cl, saturated aqueous NaHCO₃ and brine, and dried over MgSO4. The obtained mixture was filtered and concentrated in vacuo. The oily residue was dissolved in aqueous dioxane (9:1) 700 mL and heated at 80° C. in presence of catalytic PTSA until completion of starting material (˜3h). Aqueous work up of reaction mixture, followed by extraction with ethyl acetate and removal of solvent yielded 25-OH cholesterol. Crystallization was carried out in methanol to yield 25-OH cholesterol as white crystalline solid (56 g).

Yield: 56 g (89%)

M. pt: 176-178° C.

[α]²_{D}: −40° (C=1, CHCl3)

Appearance: White crystalline solid

GC analysis: 96.85% (11.04)

HPLC: 98.62% (4.38)

HNMR:

1H-NMR (400 MHz, CDCl3): δ=0.67 (s, 3H), 0.94 (s, 3H), 0.96 (d, 3H), 1.12 (s, 6H), 3.55 (m, 1H), 5.34 (d, 1H).

The 1H NMR data correspond to those known from the literature.

13C-NMR:

13C NMR (100 MHz, CDCl3): δ 140.7, 121.6, 71.7, 71.1, 56.7, 56.1, 50.1, 44.4, 42.3, 42.2, 39.7, 37.2, 36.5, 36.4, 35.7, 32.0 31.8, 31.6, 29.3, 29.1, 28.2, 24.2, 21.1, 20.7, 19.3, 18.6, 11.8;

### Example 8

### Preparation of 25-OHcholesterol (7) from (20S)-20-iodomethyl-6β-methoxy-3,5-cyclo-5α-pregnane (5)

### 1) Ethyl 1(S)-6β-methoxy-3α,5-cyclo-5α-cholesta-25-oate (6)

To a vigorously stirred mixture of 25.7 g (0.4 mol) of zinc dust and 25.7 mL (36.0 g 0.36 mol) of ethyl acrylate in 60 mL of pyridine was added 18.0 g (0.075 mol) of NiCl26H₂O. The mixture was heated to 50° C., whereupon an exotherm ensued, and stirring was continued at 65° C. for 30 min. The resulting reddish-brown mixture was cooled to 25° C. and treated during 0.5 h with a solution of 50 g (0.109 mol) of (20S)-20-iodo methyl-6β-methoxy-3α,5-cyclo-5α-pregnane (5) in 100 mL of pyridine at a rate so as to maintain the temperature below 25° C. The mixture was stirred at 25° C. for 4 h, poured into 150 mL of EtOAc, and filtered through a pad of Celite. The pad was washed with EtOAc (2×100 mL), and the filtrate and washings were washed with 1.0 N HCl (4×150 mL), 200 mL of a solution of EDTA (80.0 g EDTA+80 g NaHC03 in 1.0 L of H2O), and brine (2×100 mL), dried (Na2SO4), and evaporated to give 39 g (83%) of crude (6), which was used directly in the next step.

An analytical sample was prepared by an additional purification by silicagel column chromatography using EtOAc: n-heptane (2:98) as an eluent. Concentration of collected pure fractions yielded desired product as thick colourless oil.

Appearance: Colourless thick oil

GC purity: 97.6% (RT: 8.83)

HNMR:¹H NMR: δ 0.27 (1H, dd) 0.37 (1H, dd), 0.83 (1H, dddd), 0.87 (6H, s), 1.03 (3H, s), 1.15 (3H, t), 2.23 (1H, t), 2.23 (1H, t), 3.04 (3H, s), 4.00-4.12 (3H, 4.10 (q), 4.04 (1H dd)).

¹³CNMR:

13C NMR (100 MHz, CDCl3): δ 173.9, 82.4, 60.1, 56.5, 56.4, 55.9, 47.9, 43.3, 42.7, 40.2, 35.4, 35.3, 35.2, 35.0, 34.7, 33.3, 30.4, 28.2, 24.9, 24.1, 22.7, 21.5, 21.4, 19.2, 18.5, 14.2, 13.0, 12.2;

Mass spectrum (m/e): 431(M+1), 429(M⁻1)

### 2) 25-OH cholesterol (7)

To a stirred, cooled (ice bath) solution of 39 g (0.090 mol) of ester (6) in 200 mL of dry THF under nitrogen was added 52 mL (0.226 mol) of methyl magnesium bromide (3.0 M in ether) during 30 min. The mixture was stirred at ice bath temperature for 15 min and at room temperature for 2-3 h, cooled to 0° C., and carefully quenched with saturated NH4Cl. It was extracted with 2 L of EtOAc, washed with brine (3×250 mL), dried (Na2SO4), and evaporated in vacuo to give 39.0 g of crude product, stirred with methanol, filtered, dried to get 38 g (82%) of desired product as a color less solid which was dissolved in aqueous dioxane (9:1) 500 mL and heated at 80° C. in presence of catalytic PTSA until completion of starting material (˜3h). Aqueous work up of reaction mixture, followed by extraction with ethyl acetate and removal of solvent yielded 25-OH cholesterol. Crystallization was carried out in methanol gave 30 g of 25-OH cholesterol as white crystalline solid.

Yield: 30 g (82%)

M.pt: 175-177° C.

Appearance: White crystalline solid

GC analysis: >95%(11.04)

HPLC: ˜98% (4.38)

HNMR:

1H-NMR (400 MHz, CDCl3): 4-=0.68 (s, 3H), 0.94 (s, 3H), 0.97 (d, 3H), 1.14 (s, 6H), 3.52 (m, 1H), 5.4 (d, 1H).

The 1H NMR data correspond to those known from the literature.

### Example 9

### 25-OH cholesteryl diacetate (8)

25-OH cholesterol (56 g, 0.14 mole) was dissolved in pyridine (400 mL) followed by addition of acetic anhydride (31.36 g, 0.30 mole) and DMAP (20.0 g, 0.163 mole) at room temperature and stirred at 40° C. for 6h until completion of starting material. Reaction mixture was poured in to cold water and extracted with ethyl acetate (200 mL×4), washed the ethyl acetate layer with 1N HCl solution (50 mL×4) followed by 5% NaHCO₃ solution (200 mL), dried the solvent over anhydrous Na2SO4 and removed the solvent to get sticky solid which upon purification by column chromatography gave pure 25-OH cholesterol diacetate as white solid (50.0 g).

Yield: 50 g (75%)

M.pt: 120-124° C.

Appearance: White solid

HPLC: ˜90% (RT: 6.12)

GC analysis: 92.5% (RT: 11.3)

1HNMR:

1H-NMR (400 MHz, CDCl3): δ=0.94 (s, 3H), 0.97 (d, 3H), 1.42 (s, 6H), 2.05-2.07 (s, 6H), 2.32 (1H, dd), 2.31 (1H, dd), 4.64 (m, 1H), 5.38 (dd, 1H).

¹³CNMR:

13C NMR (100 MHz, CDCl3): δ 170.5, 170.5, 139.6, 122.6, 82.5, 73.9, 56.6, 56.1, 50.0, 42.3, 41.1, 39.7, 38.1, 36.9, 36.5, 36.1, 36.1, 35.6, 31.8, 31.8, 28.2, 27.7, 26.0, 24.2, 22.4, 21.4, 21.0, 20.4, 19.2, 18.5, 11.8;

Mass spectrum (m/e): 487(M+1)

### Example 10

### Preparation of 25-OH 7-dehydrocholesterol (9)

### 1) 25-OH 7-bromo cholesteryl diacetate

To a stirred solution of 25-OH cholesterol diacetate (50 g 0.10 mole) in 500 mL pet ether was added DDH (1,3-dibromo-5,5-dimethyl hydantoin (16.4 g, 0.057 mole) followed by catalytic perkadox (Bis(tert-butylcyclohexyl) peroxydicarbonate) as initiator. Reaction mixture was heated to reflux at 65° C., until completion of starting material, monitored by TLC and HPLC. Reaction was quenched by adding water and extracted with pet ether, washed the pet ether layer with water, dried the pet ether layer over anhydrous Na2SO4 and removed the solvent to get crude 7-bromo 25-OH cholesterol diacetate as a pale yellow thick oil (˜50 g), used as such for next reaction without further purification.

HPLC: 60% (RT 5.95)

### 2) 25-OH 7-dehydrocholesteryl diacetate

To a stirred cooled solution of 7-Bromo 25-OH cholesterol diacetate (50 g, 0.176 mole) in dry THF (200 mL) was added anhydrous TBAF (100 g, 0.317 mole) dissolved in dry THF (400 mL). Stirred the reaction mixture at 20° C. for 2 hrs. Reaction was monitored by HPLC. Reaction was quenched by adding water and extracted with ethyl acetate (3×200 mL), washed the ethyl acetate layer with brine solution (200 mL) and dried over anhydrous Na2SO4 and removed the solvent to get crude as colourless solid. The solid was stirred with methanol, filtered the solid, dried and used for saponification step.

Yield: 30 g (60%)

M.pt: 129-131° C.

Appearance: White solid

HPLC: >95% (RT: 7.18)

GC analysis: 98.8% (RT: 12.04)

1H-NMR (400 MHz, CDCl3): δ=0.94 (s, 3H), 0.95 (d, 3H), 1.42 (s, 6H), 2.05-2.07 (s, 6H), 2.53 (1H, dd), 2.39 (1H, dd), 4.74 (m, 1H), 5.39 (dd, 1H), 5.58 (dd, 1H)

¹³CNMR:

13C NMR (100 MHz, CDCl3): δ 170.5, 170.5, 141.5, 138.5, 120.2, 116.3, 82.5, 72.8, 55.9, 54.4, 46.0, 42.9, 41.1, 39.1, 37.9, 37.1, 36.6, 36.1, 36.0, 28.1, 26.1, 26.0, 22.9, 22.5, 21.4, 21.0, 20.5, 18.7, 16.2, 11.8;

Mass spectrum (m/e): 485(M+1)

### 3) 25-OH 7-dehydrocholesterol

25-OH 7-dehydrocholesteryl diacetate (30 g, 0.62 mole) was suspended in methanol (300 mL) and KOH (20 g, 0.36 mole) added and heated the reaction mixture at 45° C. for 2h. Reaction was monitored by TLC. Solvent was removed by distillation and water was added to residual mass and extracted with dichloromethane. Dichloromethane layer was washed with brine and dried over anhydrous Na2SO4, filtered and solvent was removed to get crude product. The crude product was crystallized in methanol/acetone to get pure 25-OH-7-dehydrocholesterol as white solid (25 g).

Yield: 25 g (80%)

M.pt: 187-191° C.

Appearance: White solid

HPLC: ˜98% (RT: 9.79)

HNMR:

1H-NMR (400 MHz, CDCl3): δ=0.62 (s, 3H), 0.94 (s, 3H), 0.95 (d, 3H), 1.12 (s, 6H), 3.66 (m, 1H), 5.39 (dd, 1H), 5.58(dd, 1H)

¹³CNMR:

13C NMR (100 MHz, CDCl3): δ 141.3, 139.8, 119.6, 116.3, 71.1, 70.4, 55.8, 54.5, 46.2, 44.4, 42.9, 40.8, 39.1, 38.4, 37.0, 36.4, 36.1, 32.0 29.4, 29.2, 28.1, 23.0, 21.1, 20.8, 18.8, 16.3, 11.8;

1-27. (canceled)

28. A process for preparation of 25-OH cholesterol comprising the steps of:
a) reacting (20S)-20-hydroxymethyl-6β-methoxy-3α,5-cyclo-5α-pregnane with Iodine and imidazole in presence of triphenyl phosphine to obtain (20S)-20-iodo methyl-6β-methoxy-3α,5-cyclo-5α-pregnane;
b) reacting the (20S)-20-iodo methyl-6β-methoxy-3α,5-cyclo-5α-pregnane with ethyl acrylate in presence of a reducing agent and a first base to obtain Ethyl 1(S)-6β-methoxy-3α,5-cyclo-5α-cholesta-25-oate; and
c) reacting the ethyl 1(S)-6β-methoxy-3α,5-cyclo-5α-cholesta-25-oate with methyl magnesium bromide in a solvent at room temperature under N2 followed by treating with a dehydrating agent in a solvent to obtain 25-OH cholesterol.

29. A process for preparation of 25-OH vitamin D3 (Calcifediol) which comprises;
a) preparing 25-OH cholesterol by the process of claim 28;
b) Reacting the 25-OH cholesterol with acetic anhydride in presence of a second base at 30-50° C. to obtain 25-OH cholesterol diacetate;
c) Brominating the 25-OH cholesterol diacetate with a brominating agent in presence of an initiator at 50 to 80° C. to obtain 25-OH 7-bromo cholesteryl diacetate;
d) Treating the 25-OH 7-bromo cholesteryl diacetate with TBAF in a solvent at 10 to 30° C. to obtain 25-OH 7-dehydrocholesteryl diacetate followed by subjecting the 25-OH 7-dehydrocholesteryl diacetate to alkaline hydrolysis at about 30 to 60° C. to obtain 25-OH 7-dehydrocholesterol;
e) Irradiating the 25-Hydroxy-7-dehydrocholesterol in a solvent under a high pressure mercury lamp in the presence of an irradiation sensitizer to obtain 25-hydroxy-pre vitamin D3; and
f) Heating the solution containing 25-hydroxy-pre D3 to convert 25-hydroxy-pre D3 to 25-OH vitamin D3 (Calcifediol).

30. The process as claimed in claim 28, wherein reacting the (20S)-20-iodo methyl-6β-methoxy-3α,5-cyclo-5α-pregnane with ethyl acrylate is carried out at a temperature range of 40 to 70° C.

31. The process as claimed in claim 28, wherein reacting the (20S)-20-iodo methyl-6β-methoxy-3α,5-cyclo-5α-pregnane with ethyl acrylate is carried out in the presence of the reducing agent, wherein the reducing agent comprises a combination of zinc dust and nickel chloride.

32. The process as claimed in claim 28, wherein reacting the ethyl 1(S)-6β-methoxy-3α,5-cyclo-5α-cholesta-25-oate with methyl magnesium bromide is carried out at a temperature range of 50 to 90° C.

33. The process as claimed in claim 28, wherein the first base is pyridine, DMAP, or a combination thereof.

34. The process as claimed in claim 29, wherein the second base is pyridine, DMAP, or a combination thereof.

35. The process as claimed in claim 28, wherein the dehydrating agent is para toluene sulphonic acid.

36. The process as claimed in claim 29, wherein:
the initiator is selected from the group consisting of Bis(tert-butylcyclohexyl) peroxydicarbonate, AIBN, and combinations thereof; and
the brominating agent is selected from the group consisting of DDH (1,3-dibromo-5,5-dimethyl hydantoin), NBS, and combinations thereof.

37. The process as claimed in claim 29, wherein the irradiation sensitizer is 5-(3-pyridyl)-2,2′-bithiophene, anthracene, or 9-acetyl anthracene.

38. The process as claimed in claim 29, wherein irradiating the 25-Hydroxy-7-dehydrocholesterol is carried out in a solvent selected from the group consisting of THF, diethylether, dioxane, and mixtures thereof.

39. A process for preparation of 25-OH cholesterol comprising the steps of:
a) preparing a solution of magnesium turnings in the presence of 1,2-dibromoethane and 4-bromo-2-methyl-2-[(trimethylsilyl)oxy]butane at 40 to 60° C. under N₂;
b) reacting the product of step a) with (20S)-6β-methoxy-20-(p-toluene sulfonoxymethyl)-3α, 5-cyclo-5α-pregnane and CuBr.Me2S solution to obtain 25-OH cholesterol.

40. A process for preparation of 25-OH Vitamin D3(Calcifediol), which comprises;
a) preparing 25-OH cholesterol by the process of claim 39;
b) Reacting the 25-OH cholesterol with acetic anhydride in presence of a base at 30-50° C. to obtain 25-OH cholesterol diacetate;
c) Brominating the 25-OH cholesterol diacetate with a brominating agent in presence of an initiator at 50 to 80° C. to obtain 25-OH 7-bromo cholesteryl diacetate;
d) Treating the 25-OH 7-bromo cholesteryl diacetate with TBAF in a solvent at 10 to 30° C. to obtain 25-OH 7-dehydrocholesteryl diacetate followed by subjecting the 25-OH 7-dehydrocholesteryl diacetate to alkaline hydrolysis at about 30 to 60° C. to obtain 25-OH 7-dehydrocholesterol;
e) Irradiating the 25-Hydroxy-7-dehydrocholesterol in a solvent under a high pressure mercury lamp in the presence of an irradiation sensitizer to obtain 25-hydroxy-pre vitamin D3; and
f) Heating the solution containing 25-hydroxy-pre D3 to convert 25-hydroxy-pre D3 to 25-OH vitamin D3 (Calcifediol).

41. The process as claimed in claim 40, wherein the irradiation sensitizer is 5-(3-pyridyl)-2,2′-bithiophene.

42. The process as claimed in claim 40, wherein:
the initiator is selected from the group consisting of Bis(tert-butylcyclohexyl) peroxydicarbonate, AIBN, and combinations thereof; and
the brominating agent is selected from the group consisting of DDH (1,3-dibromo-5,5-dimethyl hydantoin), NBS, and combinations thereof.

43. The process as claimed in claim 40, wherein the initiator is Bis(tert-butylcyclohexyl) peroxydicarbonate and the brominating agent is DDH.

44. A process for preparation of 25-OH-7-dehydrocholesterol, comprising:
a) reacting 25-OH cholesterol with acetic anhydride in the presence of a base at 30-50° C. to obtain 25-OH cholesterol diacetate;
b) brominating the 25-OH cholesterol diacetate with DDH (1,3-dibromo-5,5-dimethyl hydantoin) in the presence of an initiator at 50 to 80° C. to obtain 25-OH 7-bromo cholesteryl diacetate; and
c) treating the 25-OH 7-bromo cholesteryl diacetate with TBAF in a solvent at 10 to 30° C. to obtain 25-OH 7-dehydrocholesteryl diacetate; and
d) subjecting the 25-OH 7-dehydrocholesteryl diacetate to alkaline hydrolysis at about 3 to 60° C. to obtain 25-OH 7-dehydrocholesterol.

45. A compound, selected from the group consisting of:
ethyl 1(S)-6βmethoxy-3α,5-cyclo-5α-cholesta-25-oate of formula 6 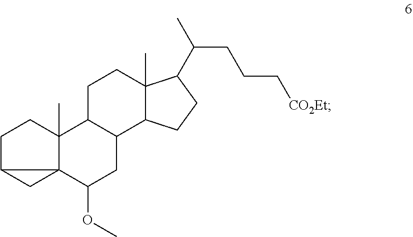 [SMILES: CCOC(=O)CCCC(C)C1CCC2C3CC(OC)C45CC4CCC5(C)C3CCC12C]
25-OH 7-dehydrocholesteryl diacetate of formula 8 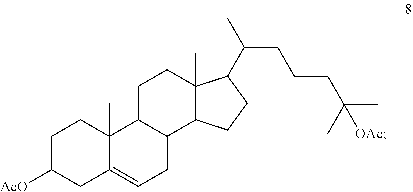 [SMILES: CC(=O)OC1CCC2(C)C(=CCC3C2CCC2(C)C(C(C)CCCC(C)(C)OC(C)=O)CCC32)C1]
and
25-OH 7-dehydrocholesterol of formula 9

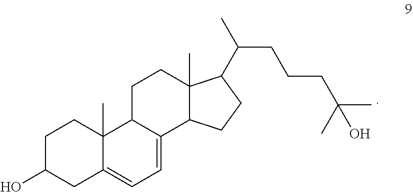 [SMILES: CC(CCCC(C)(C)O)C1CCC2C3=CC=C4CC(O)CCC4(C)C3CCC21C]

46. The compound of claim 45, which is ethyl 1(S)-6β-methoxy-3α,5-cyclo-5α-cholesta-25-oate.

47. The compound of claim 45, which is 25-OH 7-dehydrocholesteryl diacetate.

48. The compound of claim 45, which is 25-OH 7-dehydrocholesterol.
